(19) United States
(12) Patent Application Publication
(10) Pub. No.: US 2007/0173918 A1
(43) Pub. Date: Jul. 26, 2007

(54) APPARATUS AND METHODS FOR LOCATING AN OSTIUM OF A VESSEL

(76) Inventors: James H. Dreher, Santa Monica, CA (US); Amr Salahieh, Saratoga, CA (US); Jeffrey A. Krolik, Campbell, CA (US)

Correspondence Address: Vista IP Law Group LLP, 2040 MAIN STREET, 9TH FLOOR, IRVINE, CA, 92614, US

(21) Appl. No.: 11/537,569

(22) Filed: Sep. 29, 2006

Related U.S. Application Data
(60) Provisional application No. 60/722,182, filed on Sep. 30, 2005.

(51) Int. Cl. A61F 2/06

(52) U.S. Cl. 623/1.11

(57) ABSTRACT

Apparatus and methods for locating an ostium of a branch vessel include a delivery catheter having a distal end sized for introduction into the branch, and locator elements including first ends fixed to the distal end and second ends free from the distal ends. The locator elements are compressible from a transverse, deployed condition to an axial, contracted condition, wherein the second ends are disposed proximal to the first ends. During use, the catheter is directed through a guide catheter into the ostium with the locator elements compressed, and the locator elements are deployed within the branch in the contracted condition. The catheter is partially withdrawn from the branch, the locator elements resiliently expanding towards the deployed condition as they enter the main vessel. The catheter may be used to deliver a stent into the branch with the expanded locator elements facilitating positioning the stent.

This application claims benefit of U.S. provisional application Ser. No. 60/722,182, filed Sep. 29, 2005, the entire disclosure of which is expressly incorporated by reference herein.

### FIELD OF THE INVENTION

The present invention relates generally to apparatus and methods for locating an ostium of a blood vessel or other body lumen, and, more particularly, to apparatus and methods for locating an ostium of a blood vessel or other body lumen to deliver a stent or other prosthesis or perform another procedure in or adjacent the ostium.

### BACKGROUND

Tubular endoprosthesis or “stents” have been suggested for dilating or otherwise treating stenoses, occlusions, and/or other lesions within a patient's vasculature or other body lumens. For example, a self-expanding stent may be maintained on a catheter in a contracted condition, e.g., by an overlying sheath or other constraint, and delivered into a target location, e.g., a stenosis within a blood vessel or other body lumen. When the stent is positioned at the target location, the constraint may be removed, whereupon the stent may automatically expand to dilate or otherwise line the vessel at the target location. Alternatively, a balloon-expandable stent may be carried on a catheter, e.g., crimped or otherwise secured over a balloon, in a contracted condition. When the stent is positioned at the target location, the balloon may be inflated to expand the stent and dilate the vessel.

Sometimes, a stenosis or other lesion may occur at an ostium or bifurcation, i.e., where a branch vessel extends from a main vessel. For example, such a lesion may form within a coronary artery immediately adjacent the aortic root. U.S. Pat. No. 5,749,890 to Shaknovich discloses a stent delivery assembly for placing a stent in an ostial lesion. U.S. Pat. No. 5,632,762 to Myler discloses a tapered balloon on a catheter for positioning a stent within an ostium. U.S. Pat. No. 5,607,444 to Lam discloses an expandable ostial stent including a tubular body and a deformable flaring portion. Published application US 2002/0077691 to Nachtigall discloses a delivery system that includes a sheath for holding a stent in a compressed state during delivery and a retainer that holds a deployable stop in an undeployed position while the delivery system is advanced to a desired location.

Accordingly, apparatus and methods for locating an ostium and/or for delivering a stent within an ostium would be useful.

### SUMMARY OF THE INVENTION

The present invention is directed to apparatus and methods for locating a branch body lumen extending from a main body lumen, and, more particularly, to apparatus and methods for locating an ostium or bifurcation of a blood vessel or other body lumen, e.g., for delivering a stent or other prosthesis within or adjacent the ostium, for accessing the blood vessel, and/or for performing another procedure at, within, or beyond the bifurcation.

In accordance with one embodiment, an apparatus is provided for locating an ostium of a body lumen that includes a tubular member including a proximal end, a distal end sized for introduction into a body lumen, and a lumen extending between the proximal and distal ends, and an elongate member including a distal portion disposed within the lumen such that the distal portion may be advanced beyond the tubular member distal end. One or more locator elements are provided on the distal portion, each locator element including a first end fixed to the distal portion and a second end free from the distal portion, each locator element being resiliently compressible to a contracted condition when the distal portion is disposed within the lumen such that the second end is disposed proximal to the first end, each locator element being resiliently expandable to an enlarged condition when fully deployed.

In exemplary embodiments, the locator element(s) may include a wire, a band, and/or a loop. Optionally, an expandable support may be provided adjacent the locator element(s) for supporting the locator element in the enlarged condition. Optionally a tubular prosthesis, e.g., a stent, may be provided on the distal portion, e.g., adjacent the one or more locator elements.

In accordance with another embodiment, an apparatus is provided for locating an ostium of a body lumen that includes an elongate member including a distal portion disposed within the lumen such that the distal portion may be advanced beyond the tubular member distal end, and one or more locator elements on the distal portion. The locator element(s) may be resiliently compressible to a contracted condition, e.g., an axial orientation adjacent the distal portion, and/or resiliently expandable to an enlarged condition, e.g., a transverse, lateral, or other non-axial orientation to facilitate locating an ostium.

In one embodiment the locator element includes a loop on the distal portion, e.g., supported eccentrically relative to the distal portion in the enlarged condition. For example, the locator element may include a loop disposed eccentrically around the distal portion, e.g., supported by one or more supports. In another embodiment, the locator element may include a locator loop oriented laterally away from the distal portion in the enlarged condition. For example, the locator loop may extend laterally from one side of the distal portion, and a constraining loop may extend laterally from an opposite side of the distal portion to limit deflection of the locator loop.

In accordance with still another embodiment, a method is provided for locating an ostium communicating from a main body lumen to a branch body lumen. A distal end of a delivery catheter may be advanced into the main body lumen, the distal end including one or more locator elements constrained in a contracted condition. The distal end may be advanced into the ostium until the one or more locator elements are disposed within the branch body lumen. The one or more locator elements may be at least partially released within the branch body lumen, and the distal end may be partially withdrawn until the one or more locator elements at least partially emerge from the ostium and expand towards an enlarged condition. A procedure may be performed at or within the ostium based upon the position of the one or more locator elements in the enlarged condition. For example, a stent may be delivered within at least one of the ostium and the branch upon positioning the stent using the one or more locator elements.

In accordance with yet another embodiment, a method is provided for delivering a stent within an ostium communicating from a main body lumen to a branch body lumen. A distal end of a delivery catheter may be advanced into the main body lumen, the distal end including one or more locator elements constrained in a contracted condition. The distal end may be advanced into the ostium until the one or more locator elements are disposed within the branch body lumen, and the one or more locator elements may be released within the branch body lumen. The distal end may be partially withdrawn until the one or more locator elements at least partially emerge from the ostium and expand towards an enlarged condition.

Optionally, the one or more locator elements may automatically withdraw the distal end to accommodate expansion of the one or more locator elements towards the enlarged condition. In addition or alternatively, the delivery catheter may retracted and/or advanced to position the distal end at a desired location relative to the ostium using the one or more locator elements. A procedure, e.g., stent delivery, may be performed at or within the ostium based upon the position of the one or more locator elements in the enlarged condition.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIGS. 1 and 2 are perspective views of an apparatus for delivering a stent, including a guide catheter and a delivery catheter, the delivery catheter having a distal end carrying a locator adjacent the stent in contracted and enlarged conditions, respectively. FIGS. 3-9 are cross-sectional views of a patient's body, showing a method for positioning and/or delivering a stent within an ostium of a body lumen using the apparatus of FIGS. 1 and 2.

FIG. 5A is a detail of FIG. 5, showing the locator being exposed from the guide catheter.

FIG. 10 is a cross-sectional view of a patient's body, showing an alternative embodiment of an apparatus for positioning a stent within an ostium.

FIGS. 11-14 are cross-sectional views of a patient's body, showing other alternative embodiments of apparatus for positioning a stent within an ostium.

FIG. 15 is a cross-sectional view of a patient's body, showing yet another alternative embodiment of an apparatus for positioning a stent within an ostium.

FIG. 16 is a perspective view of still another embodiment of an apparatus for positioning a stent within an ostium.

FIGS. 16A and 16B are top and side views, respectively of the apparatus of FIG. 16.

### DETAILED DESCRIPTION OF THE EXEMPLARY EMBODIMENTS

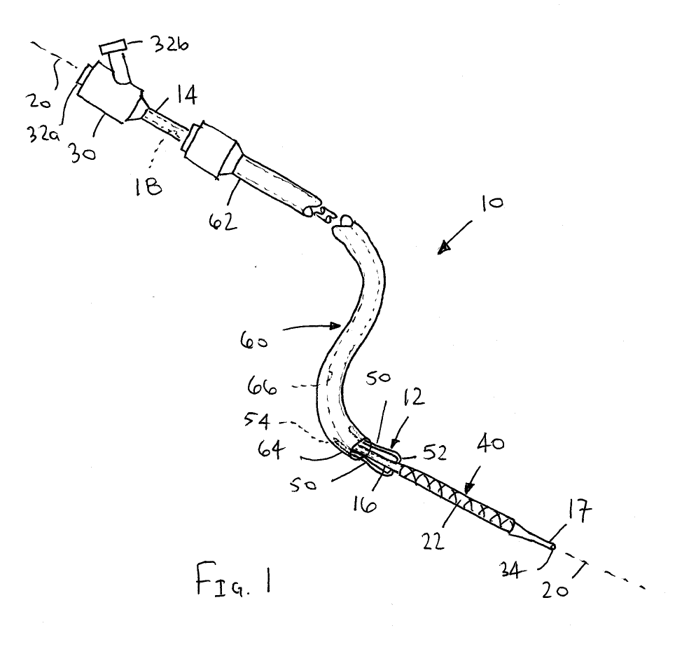

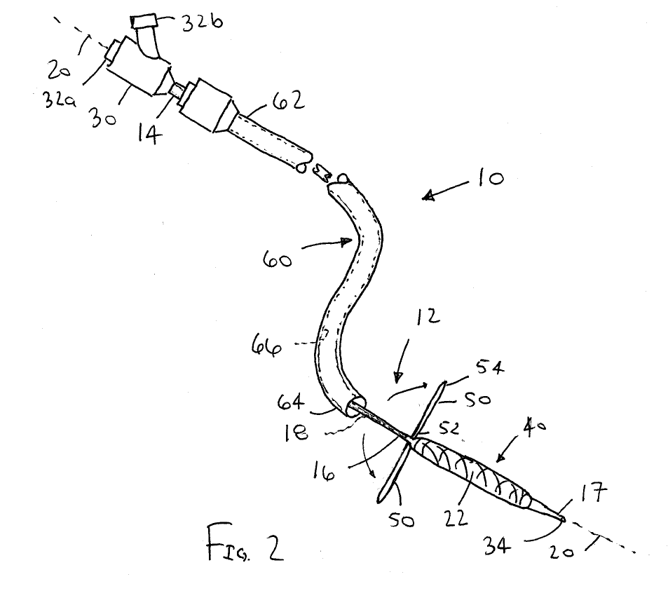

Turning to the drawings, FIGS. 1 and 2 show an exemplary embodiment of an apparatus 10 for delivering a stent or other prosthesis 40, e.g., into an ostium or other bifurcation between a main lumen and a branch lumen (not shown). Generally, the apparatus 10 includes a catheter or other elongate tubular member 12 having a proximal end 14, a distal end 16, and one or more lumens 18 extending between the proximal and distal ends 14, 16, thereby defining a longitudinal axis 20 between the proximal and distal ends 14, 16. The delivery catheter 12 includes one or more locator elements 50 on the distal end 16, e.g., proximal or otherwise adjacent to a stent 40 also carried on the distal end 16.

Optionally, one or more balloons or other expandable members 22 may be provided on the distal end 16 of the delivery catheter 12 for expanding and/or deploying the stent 40. In addition, the distal end 16 may include one or more markers, e.g., one or more bands of radiopaque material (not shown), to facilitate positioning the delivery catheter 12. In addition or alternatively, the delivery catheter 12 may include one or more therapeutic and/or diagnostic elements (not shown) on the distal end 16, e.g., instead of or in addition to the stent 40 and/or balloon(s) 22.

In addition, the apparatus 10 may include a guide catheter 60 including a proximal end 62, a distal end 64, and a lumen 66 extending therebetween. The distal end 64 may be sized and/or shaped to facilitate advancement into a patient's vasculature or other body lumen, as described further below. The lumen 66 may have sufficient size for receiving the distal end 16 of the delivery catheter 12 therethrough, e.g., with the locator element(s) 50 in a contracted condition, also as explained further below. Optionally, the distal end 64 of the guide catheter 60 may be biased to a predetermined shape, e.g., a “J” shape, which may facilitate positioning the guide catheter 60 within or adjacent an ostium. The guide catheter 60 may be constructed from substantially flexible and/or floppy materials, e.g., plastic having a braid or other reinforcement (not shown) that sufficiently supports the guide catheter 60 to prevent kinking or buckling, while allowing the guide catheter 60 to be directed easily through tortuous anatomy.

Optionally, the apparatus 10 may include other components to provide a system or kit for delivering the stent 40, e.g., a sheath that may be advanced over and/or retracted from the distal end 16 of the delivery catheter 12, one or more syringes or other sources of inflation media and/or vacuum, tubing, and/or one or more guidewires (all not shown).

With continued reference to FIGS. 1 and 2, the delivery catheter 12 may be formed from one or more tubular bodies, e.g., having variable flexibility along its length. For example, the distal end 16 may be substantially flexible to facilitate insertion through tortuous anatomy, e.g., terminating in a rounded, tapered, and/or other substantially atraumatic distal tip 17. The distal end 16 may be sized and/or shaped for introduction into a body lumen, e.g., having a diameter between about one and seven millimeters (1-7 mm), or less than 1.5 millimeters. The proximal end 14 may be substantially flexible or semi-rigid, e.g., having sufficient column strength to facilitate advancing the distal end 16 through a patient's vasculature by pushing on the proximal end 14. The delivery catheter 12 may be formed from plastic, metal, or composite materials, e.g., a plastic material having a wire, braid, or coil core, which may prevent kinking or buckling of the delivery catheter 12 during advancement.

As shown, the delivery catheter 12 may include a handle 30 on the proximal end 14, e.g., to facilitate manipulating the delivery catheter 12. The handle 30 may include one or more ports 32 communicating with respective lumens 18 within the delivery catheter 12. The handle 30 may be molded, machined, or otherwise formed from plastic, metal, or composite material, e.g., providing an outer casing, which may be contoured or otherwise shaped to ease manipulation. The proximal end 14 of the delivery catheter 12 may be attached to the handle 30, e.g., by bonding, cooperating connectors, interference fit, and the like. Optionally, if the apparatus includes any actuatable components (not shown) on the distal end 16, the handle 30 may include one or more actuators (not shown), such as one or more slides, dials, buttons, and the like, for actuating or otherwise manipulating the components from the proximal end 14.

In the embodiment shown in FIGS. 1 and 2, the delivery catheter 12 includes at least two lumens 18 extending between the proximal ends 14, 16. For example, the delivery catheter 12 may include a guidewire or instrument lumen that extends from a port 32a in the handle 30 to an opening 34 in the distal tip 17. The instrument lumen may have sufficient size to allow a guidewire or other rail or instrument (not shown) to be inserted therethrough, e.g., to facilitate advancing the delivery catheter 12 over the rail, as explained further below. Optionally, the handle 30 may include one or more seals (not shown) within or adjacent the port 32a, e.g., a hemostatic seal that prevents fluid, such as blood, from flowing proximally out of the port 32a, yet allows one or more instruments to be inserted therethrough and into the instrument lumen.

In addition, the delivery catheter 12 may include one or more inflation lumens that extend from respective side port(s) 32b in the handle 30 through the delivery catheter 12 to openings (not shown) that communicate with an interior of a respective balloon 22. The side port(s) 32b on the handle 30 may include one or more connectors, e.g., a luer lock connector (not shown), one or more seals (also not shown), and the like. A source of inflation media and/or vacuum, e.g., a syringe filled with saline (not shown), may be connected to the side port(s) 32b, e.g., via tubing (also not shown), for expanding and/or collapsing the balloon(s) 22.

As shown in FIGS. 1 and 2, the delivery catheter 12 includes one balloon 22 on the distal end 16. Alternatively, the delivery catheter 12 may include multiple balloons (not shown) on the distal end 16 over which the stent 40 may be placed. Additional information on multiple balloon catheters that may be provided and methods for using them are disclosed in co-pending applications Ser. No. 11/136,266, filed May 23, 2005, and 60/727,703, filed Oct. 17, 2005, the entire disclosures of which are expressly incorporated by reference herein.

The balloon (or balloons, not shown) 22 may be bonded or otherwise secured to the distal end 16 of the delivery catheter 12. For example, ends of the balloon 22 may be attached to the distal end 16 using one or more of bonding with an adhesive, sonic welding, an annular collar or sleeve, and the like. The balloon 22 may be expandable from a contracted condition (e.g., as shown in FIG. 1), which may facilitate advancement through a patient's vasculature to an enlarged condition for expanding or otherwise deploying the stent 40 (e.g., as shown in FIG. 2).

The balloon 22 may be formed from substantially inelastic material, e.g., PET, nylon, or PEBAX, such that the balloon 22 expands to a predetermined size in its enlarged condition once sufficient fluid is introduced into the interior of the balloon 22. Alternatively, the balloon 22 may be formed from substantially elastic material, e.g., silicone, polyurethane, or polyethylene, such that the balloon 22 may be expanded to a variety of sizes depending upon the volume and/or pressure of fluid within the interior.

The stent 40 may be formed from a variety of materials that may be plastically deformed to allow expansion of the stent 40. For example, the stent 40 may be formed from metal, such as stainless steel, tantalum, MP35N, Niobium, Nitinol, and cobalt chromium (such as L605), plastic, or composite materials. In particular, the materials of the stent 40 may be plastically deformed under the pressures experienced when the balloon 22 is expanded such that all or one or more portions of the stent 40 are deformed beyond their elastic limit. Thus, when the balloon 22 is subsequently collapsed, the stent 40 may maintain its expanded configuration with minimal recoil. For example, the stent 40 material may resist collapsing back towards its reduced configuration if the tissue surrounding the body lumen attempts to constrict or otherwise return to its occluded shape.

Alternatively, at least a portion of the stent 40 may be self-expanding. For example, the stent 40 may be biased to expand at least partially outwardly yet may be constrained over the balloon 22 in a contracted condition to facilitate delivery, e.g., using a sheath, filament, and the like (not shown). In this alternative, the stent 40 may be formed from Nitinol or other shape memory or superelastic materials. Optionally, the resistance of the stent 40 to expansion may be varied along its length. This performance of the stent 40 may be based upon mechanical properties of the material, e.g., which may involve heat treating one or more portions of the stent 40 differently than other portions. In addition or alternatively, the structure of the stent 40 may be varied, e.g., by providing struts, fibers, or other components in different portions having different widths, thicknesses, geometry, and the like.

The stent 40 may be a generally tubular structure, e.g., including openings in a tubular wall that facilitate expansion of the stent 40 and/or allow tissue ingrowth. For example, the stent may be an elongate tube that has slots or other openings formed in the tube wall, e.g., by laser cutting, mechanical cutting, chemical etching, machining, and the like. Alternatively, the stent 40 may be a braided or other structure, e.g., formed from one or wires or other filaments braided or otherwise wound in a desired manner. Additional possible stent structures may include helical coil wires or sheets, welding or otherwise attaching wire or other structures together, and the like. If desired, one or more portions of the stent 40 may include a membrane, film, or coating (not shown), e.g., to create a nonporous, partially porous, or porous surface between cells of the stent 40 and/or to carry one or more therapeutic compounds. Additional information on stents that may be provided are disclosed in co-pending applications Ser. No. 11/439,717, filed May 23, 2006, Ser. No. 11/466,439, filed Aug. 22, 2006, and 60/731,568, filed Oct. 28, 2005, the entire disclosures of which are expressly incorporated by reference herein.

With continued reference to FIGS. 1 and 2, each locator element 50 is a deflectable or otherwise expandable member including a first end 52 fixed to the distal end 16 of the delivery catheter 12, and a second end 54 free from the distal end 16. In a first embodiment, each locator element 50 may be a wire structure, e.g., a section of flat or round cross-section wire that terminates in a substantially atraumatic tip on the second end 54. Alternatively, the locator elements 50 may be formed from other structures, such as a flange or other flat sheet of material having one end attached to the distal end 16 of the delivery catheter 12.

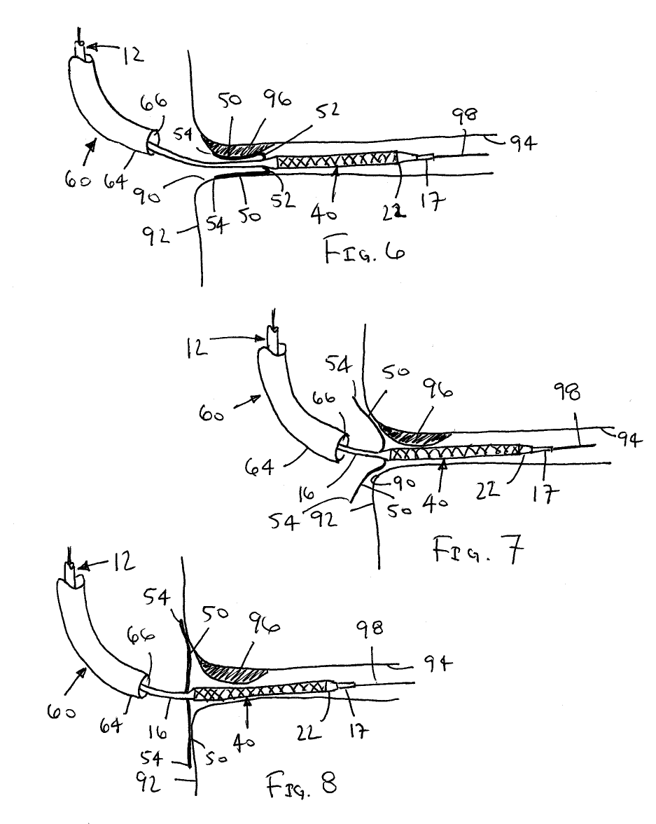

Turning to FIG. 11, in an alternative embodiment, locator elements 150 may be provided that are formed from loops of material, e.g., including first and second resilient struts 151 extending from the distal end 116 of the catheter 112, e.g., defining the first end 152. As shown, a curved intermediate region 156 extends between the first and second struts 151, thereby defining the second free end 154. The locator elements 150 may be formed from a single strand of wire defining the first strut 151, the intermediate region 156, and the second strut 151. The wire may be heat treated, plastically deformed, or otherwise treated to bias a desired shape into the individual locator elements 150, e.g., to define an elongated loop shape.

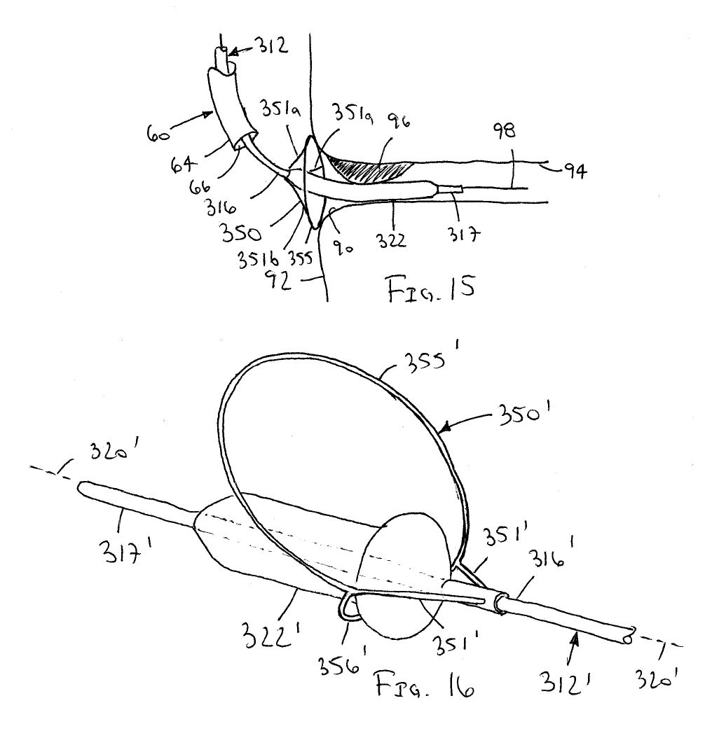

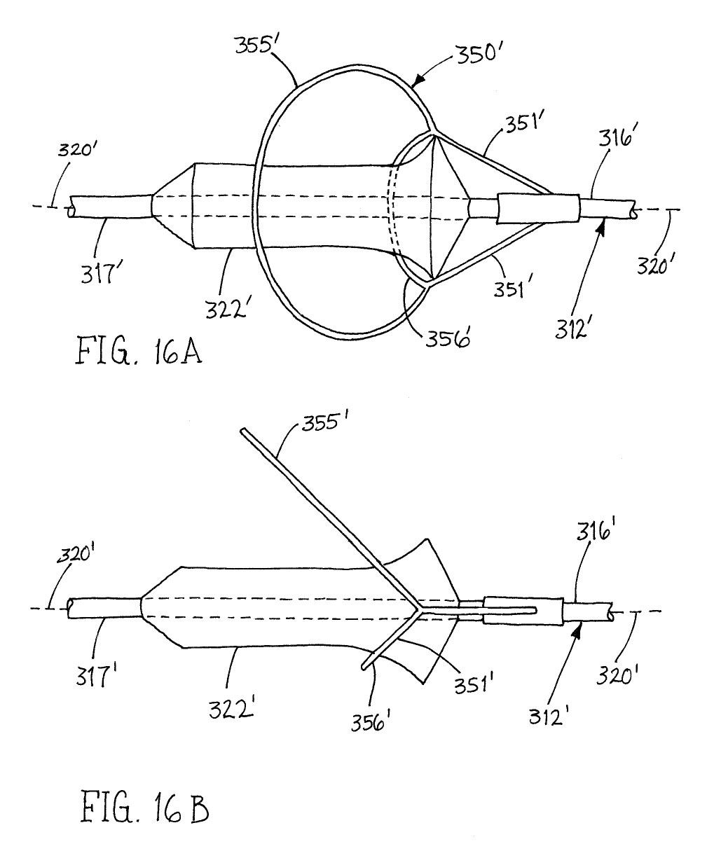

FIGS. 15 and 16 show still other embodiments of a locator element 350, 350′ that may be provided on a catheter 312, 312,′ as described further elsewhere herein. Other embodiments of locator elements that may be provided are also disclosed in co-pending application Ser. No. 11/419,997, filed May 23, 2006, the entire disclosure of which is expressly incorporated by reference herein.

Returning to FIGS. 1 and 2, the locator element(s) 50 (which may be any of the embodiments described herein) may be formed from an elastic or superelastic material, e.g., metal such as Nitinol, stainless steel, and the like, plastic, and/or composite materials (e.g., a metal wire core covered with a plastic coating). Optionally, the locator element(s) 50 may be formed from radiopaque material or may have one or more radiopaque markers, e.g., on the second end 54. For example, a radiopaque coil may be wound around, soldered, and/or otherwise attached to one or more portions of the locator elements(s) 50. Alternatively, all or one or more portions of the locator element(s) 50 may be coated or otherwise have radiopaque material applied thereto to facilitate observation of the locator element(s) 50, e.g., using fluoroscopy or other external imaging.

The locator element(s) 50 may be generally resiliently compressible, e.g., folded or deflected, to a contracted condition, such as that shown in FIG. 1, and resiliently biased to expand to an enlarged or transverse condition, such as that shown in FIG. 2, when free from external forces. For example, the locator element(s) 50 may be compressed against the distal end 16 of the delivery catheter 12 and constrained in the contracted condition, e.g., when the distal end 16 of the delivery catheter 12 is loaded into the lumen 66 of the guide catheter 60. In this condition, the locator elements 50 may extend substantially axially along and/or adjacent the distal end 16, e.g., proximally away from the stent 40. When the distal end 16 of the delivery catheter 12 is advanced beyond the distal end 64 of the guide catheter 60 (or the guide catheter 60 is retracted), the locator element(s) 50 may resiliently expand to the enlarged condition.

Alternatively, an external sheath, sleeve, or other tubular member (not shown) may be provided that extends over the distal end 16 of the delivery catheter 12 to constrain the locator element(s) 50. In one embodiment, such a sheath may also cover the stent 40 and/or balloon 22 during introduction into a patient's body. Thus, the sheath may have a length greater than the guide catheter 60 such that the sheath may be retracted from the proximal end 14 of the catheter 12, e.g., to expose the locator element(s) 50 and/or stent 40. Alternatively, the sheath may have a relative short length, e.g., sufficient to cover the distal end 16, and may be used to load the delivery catheter 12 into the proximal end 62 of the guide catheter 60, whereupon the sheath may be removed.

Although the locator element(s) 50 may be biased towards the enlarged condition, the locator element(s) 50 may be substantially atraumatic. For example, when the locator element(s) 50 are released from the contracted condition, e.g., within a branch blood vessel, as described below, the outward spring force or bias of the locator element(s) 50 may be relatively soft, i.e., sufficient to bias the locator element(s) 50 to contact the wall of a vessel, but insufficient to drive the locator element(s) 50 against the wall of a vessel to scive, puncture, damage or otherwise bear substantially against the wall of the vessel. Thus, the if the locator element(s) 50 are deployed within a relatively small space, e.g., within a branch vessel, the locator element(s) 50 may simply expand until they gently contact the wall of the vessel. If the locator element(s) 50 are translated into a larger space, e.g., a main vessel adjacent the branch vessel, the locator element(s) 50 may be free to expand fully towards the enlarged condition without damaging the vessel wall.

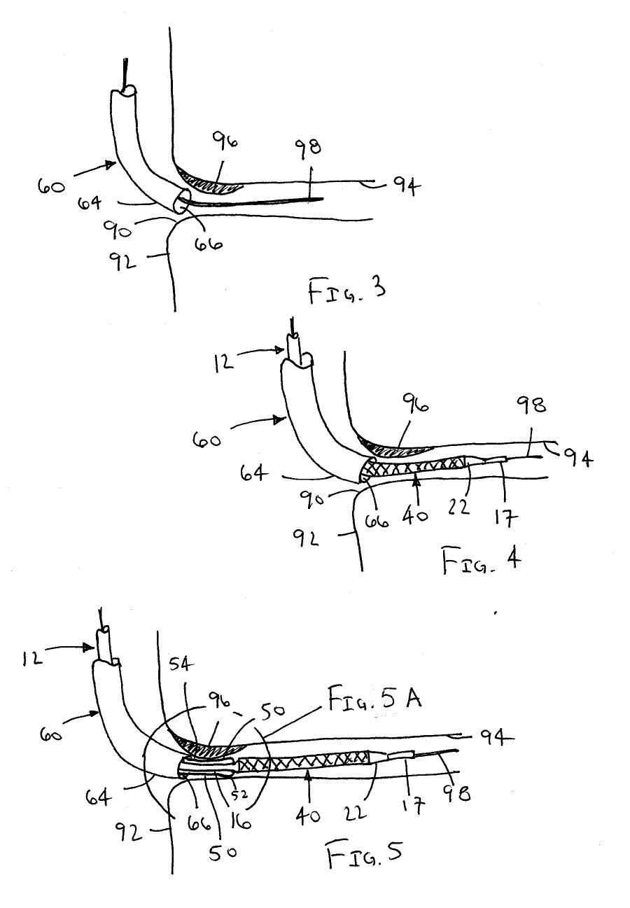

With additional reference to FIG. 5A, the first end 52 of each locator element 50 may be attached or otherwise secured to the distal end 16 of the delivery catheter 12. For example, an adhesive, sonic welding, fusing, and the like may be used to bond the first end(s) 52 to the surface of the distal end 16. In addition or alternatively, a band of material, e.g., a heat shrink tube or other band of plastic, metal, wire, and the like, may be wrapped or otherwise extend around the first end(s) 52 of the locator element(s) 50. In addition or alternatively, the first end(s) 52 of the locator element(s) 50 may be at least partially embedded into the delivery catheter 12, e.g., into slots or holes (not shown) partially or completely penetrating the wall of the delivery catheter 12. In yet another alternative, the first end(s) 52 may be part of an annular band (also not shown) that may crimped or otherwise secured around the delivery catheter 12, e.g., in addition or instead the other attachment methods described above. In this alternative, the first end(s) 52 of multiple locator element(s) 50 may be fixed to the annular band, e.g., spaced apart around a circumference of the band in a symmetrical or asymmetrical arrangement. The locator elements 50 may be biased such that the locator elements 50 extend transversely relative to the longitudinal axis 20 of the delivery catheter 12 in the enlarged condition. For example, the locator elements 50 may transition from an axial direction, e.g., where the locator elements 50 are constrained adjacent to the delivery catheter 12, as shown in FIG. 5A, to a transverse direction, when free from external forces, as shown in FIG. 2. As shown in FIG. 2, the locator elements 50 may be biased to extend substantially perpendicular to the longitudinal axis 20 in the enlarged condition. In alternative embodiments, the locator elements 50 may be biased to extend laterally relative to the longitudinal axis 20, e.g., defining an acute or oblique angle with the longitudinal axis 20.

As shown in FIG. 2, the locator elements 50 may be biased to a substantially straight shape. Alternatively, the locator elements may be biased towards other shapes in the enlarged condition. For example, as shown in FIG. 12, a delivery catheter 112′ is shown that includes locator elements 150′ defining curved loops on its distal end 116.′ Unlike the embodiment shown in FIG. 11, the locator elements 15040 0 include a first end 152′ fixed to the distal end 116′ and a second free end 154′ that is biased to curve back towards the first end 152,′ thereby defining an open loop or other curved shape. When this embodiment is constrained in the contracted condition, the second ends 154′ may extend axially, e.g., proximally, relative to the first ends 152.′ When the locator elements 150′ are deployed, the second ends 154′ may be free to slide or move along or otherwise relative to the distal end 116′as the locator elements 150′ expand towards the enlarged condition. The curved shape may enhance resistance of the locator elements 150′ to be deflected from the enlarged condition, e.g., providing a greater tactile feedback to the user.

The locator elements 150′ may be formed similar to the locator elements 50 described with reference to FIGS. 1 and 2, e.g., from a band of material rolled or otherwise biased along a width of the band to the curved shape. Alternatively, the locator elements 150′ may be formed from one or more wires formed similar to the locator elements 150 described with reference to FIG. 11, except that the locator elements 150′ may be rolled or otherwise biased to the curved shape.

Returning to FIGS. 1 and 2 (although applicable to all embodiments described herein), the locator element(s) 50 may have sufficient strength (e.g., column strength and/or bending resistance) to be self-supporting, yet be at partially deflectable, e.g., to provide tactile feedback to a user, as explained further below. For example, one or more portions of the locator element(s) 50 may bend or flex when the locator element(s) 50 contact and/or are pushed against a surface (e.g., a wall of a body lumen adjacent an ostium). The contact may provide an initial tactile feedback, and thereafter resist further bending or flexing providing a second or additional tactile feedback.

However, if it becomes necessary, the bias of the locator element(s) 50 may be overcome, e.g., by pushing or pulling the delivery catheter 12. For example, if for some reason, it becomes necessary to abort a procedure or otherwise remove the delivery catheter 12 and locator element(s) 50 from the site, the locator element(s) 50 may be sufficiently flexible to bend or otherwise yield when sufficient force is applied without substantial risk of damage to the patient.

The second ends 54 of the locator elements 50 (or any of the other embodiments described herein) may be rounded or otherwise substantially atraumatic, e.g., to prevent damaging a vessel wall during deployment and/or manipulation of the catheter 12. In one embodiment, radiopaque coils (not shown) on or adjacent the second ends 54 of the locator elements 50 may prevent puncturing, sciving, or otherwise damaging a vessel wall contacted by the second ends 54. Alternatively, other atraumatic tips may be provided on the second ends 54, similar to those disclosed in application Ser. No. 11/419,997, incorporated by reference herein.

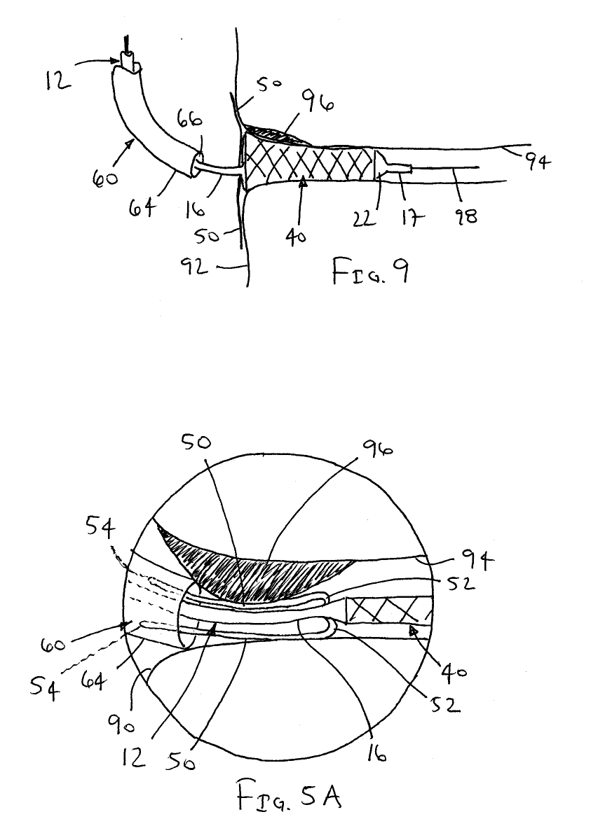

Optionally, structures may be provided on the distal end 16 of the delivery catheter 12 to direct and/or bias the locator element(s) 50 towards a desired configuration, e.g., to maintain the locator element(s) 50 in the enlarged condition. For example, as shown in FIG. 10, the delivery catheter 12′ may include an expandable member, e.g., a balloon 58,′ adjacent the locator element(s) 50,′ e.g., immediately proximal to the first end(s) 52′ of the locator element(s) 50.′ The balloon 58′ may be selectively expanded, e.g., by delivering inflation media from a side port in the handle 30 (not shown, see, e.g., FIGS. 1 and 2), through an inflation lumen, into an interior of the balloon 58.′ When inflated, the balloon 58′ may contact the locator element(s) 50,′ thereby supporting the locator element(s) 50.′ Thus, the balloon 58′ may enhance resistance of the locator element(s) 50′ being directed proximally and/or back towards the contracted condition, e.g., when the expanded locator element(s) 50′ are directed against a wall surrounding an ostium, as described further below.

Alternatively, other structures may be provided on the distal end 16 that may be selectively actuated to support the locator element(s) 50. For example, an expandable frame or other mechanical structure (not shown) may be provided that may be selectively expanded by a slider or other actuator (also not shown) on the handle 30.

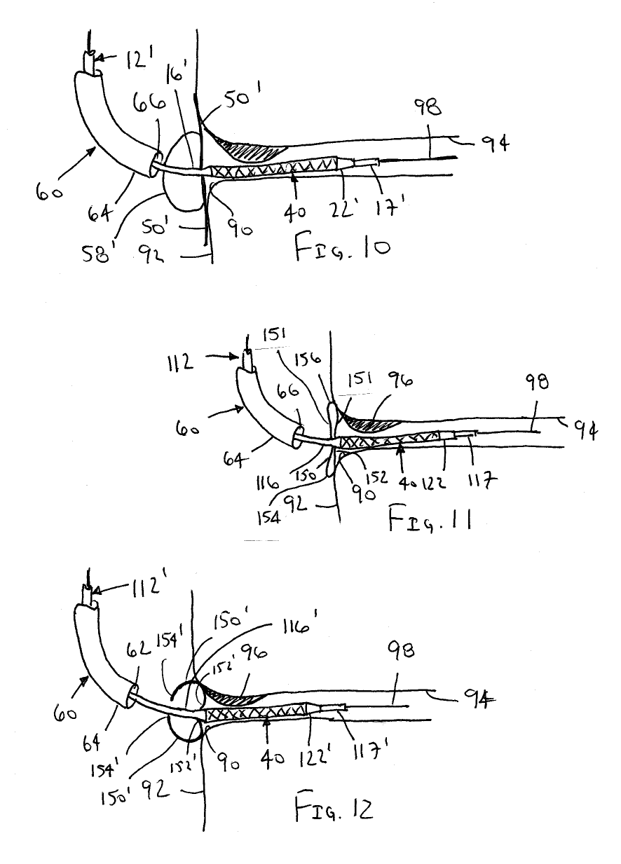

In other embodiments, the locator elements may be selectively expandable and/or collapsible. For example, as shown in FIG. 13, a delivery catheter 212 is shown that includes locator wires or other elements 250, constructed similar to those described elsewhere herein, a stent 40, and/or balloon 222. In addition, the delivery catheter 212 includes a sheath or other structure 260 including a distal end 264 slidable along the distal end 216 towards and/or away from the stent 40 and/or balloon 222. The locator wires 250 may include first ends 252 fixed to a distal end 216 of the delivery catheter 212, e.g., adjacent a stent 40 and/or balloon 222, as described above, and second ends 254 fixed to the distal end 264 of the sheath 260. Thus, the second ends 254 may be selectively movable relative to the first ends 252 to direct the locator wires 250 between contracted and enlarged conditions.

For example, the locator wires 250 may be directed to the contracted condition by directing the sheath 260 proximally, thereby pulling the second ends 254 distally away from the first ends 252. This creates an axial tension in the locator wires 250 that deforms the locator wires 250 against the distal end 216 of the delivery catheter 212. Alternatively, the sheath 260 may be rotated about its axis, thereby winding the locator wires 250 around the delivery catheter 212. When it is desired to expand the locator wires 250 towards the enlarged condition, the sheath 260 may be directed distally or rotated to release the locator wires 250, which may resiliently expand towards the enlarged condition.

The locator wires 250 may have a cross-section designed to bias the locator wires 250 expanding to a desired orientation in the enlarged condition. For example, the locator wires 250 may be formed from one or more flat wires with a width disposed against the catheter 212 and a thickness oriented transversely away from the catheter 212 in the contracted condition, the thickness being smaller than the width. Thus, as the ends 252, 254 are directed towards one another, the flat wire(s) may be biased to curve or deflect transversely away from the catheter 212 within a plane, as shown in FIG. 13, to define the enlarged condition.

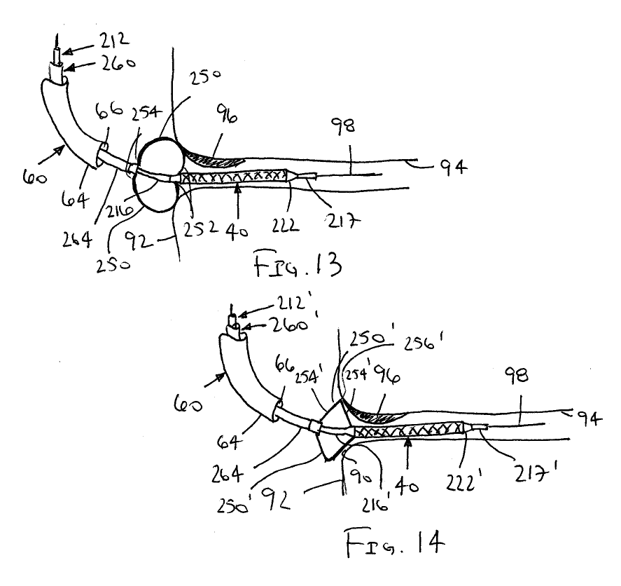

As shown in FIG. 13, the locator wires 250 may be biased to a circular or other curved shape in the enlarged condition. In addition or alternatively, the locator wires 250′ may be biased into a shape including one or more straight sections. For example, as shown in FIG. 14, the locator wires 250′ may be biased to an “L” or “V” shape including substantially straight struts 254′ connected by an intermediate curved region 256.′ Thus, when released from external forces, the locator wires 250′ may resiliently assume the enlarged condition shown. Alternatively, the locator wires 250′ may include a weakened intermediate region 256′ such that the locator wires 250′ may be compressed axially to cause the intermediate region 256′ to buckle outwardly, thereby causing the struts 254′ to deflect outwardly. The locator wires 250′ may then be compressed again towards the contracted condition, e.g., by manipulating the sheath 260,′ as described above.

Turning to FIGS. 3-8, an exemplary method is shown for using the apparatus 10 (which may be any of the embodiments described herein) to deliver a stent 40 into an ostium 90. The ostium 90 may be an opening in a wall of a first or main body lumen or trunk 92 that communicates with a second body lumen or branch 94. In an exemplary embodiment, the trunk 92 may be the aortic root and the branch 94 may be a coronary artery. In another embodiment, the trunk 92 may be the distal aorta, and the branch 94 may be a renal artery or other abdominal branch. It will be appreciated that the apparatus and methods described herein may be applicable perpendicular, from another body lumen or trunk, e.g., within a patient's vasculature or other systems.

As shown in FIG. 3, an occlusion or other lesion 96 may exist at and/or adjacent to the ostium 90, e.g., extending at least partially into the branch 94. For example, the lesion 96 may other fluid flow between the trunk 92 and the branch 94.

Initially, as shown in FIG. 3, a guidewire 98 or other rail may be introduced from the trunk 92 through the ostium 90 into the branch 94. As shown, the lesion 96 at the ostium 90 partially occludes the ostium 90 and extends into the branch 94. The guidewire 98 may be placed using conventional methods. For example, a percutaneous puncture or cut-down may be created at a peripheral location (not shown), such as a femoral artery, carotid artery, or other entry site, and the guidewire 98 may be advanced through the patient's vasculature from the entry site, e.g., alone or with the aid of guide catheter 60. If the lesion 96 completely occludes the branch 94, the guidewire 98 may be directed through the occlusion or other devices (not 20 shown) may be advanced over the guidewire 98 or otherwise in conjunction with the guidewire 98 to create a passage through the lesion 96 for the guidewire 98.

After the guidewire 98 is directed into the branch 94 beyond the lesion 96, it may be desirable to at least partially dilate the lesion 96. For example, an angioplasty catheter (not shown) may be advanced through the guide catheter 60 and/or over the guidewire 98 into and through the lesion 96, whereupon a balloon or other element on the catheter may be expanded to at least partially dilate the lesion 96. If desired, other procedures may also be performed at the lesion 96, e.g., to soften, remove, or otherwise treat plaque or other material forming the lesion 96, before the stent 40 is implanted. After completing any such procedures, instruments advanced over the guidewire 98 may be removed.

As shown in FIG. 3, the distal end 64 of the guide catheter 60 has been advanced over the guidewire 98 into the trunk 92, e.g., until the distal end 64 is disposed adjacent or proximal to the ostium 90. The guide catheter 60 may be used to advance one or more instruments (such as those just described) over the guidewire 98 and into the trunk 92 and/or branch 94.

Turning to FIG. 4, a distal end 16 of the delivery catheter 12 may be advanced over the guidewire 98 and through the lumen 66 of the guide catheter 60 from the entry site into the trunk 92. As can be seen in FIGS. 5 and 5A, the locator elements 50 are carried in the contracted condition through the guide catheter 60 with the second ends 54 disposed proximal to the first ends 52. In one embodiment, the distal tip 17 may be loaded into the proximal end 62 (not shown, see, e.g., FIGS. 1 and 2) of the guide catheter 60 with the locator elements 50 expanded. The distal end 16 may be advanced into the lumen 66 until the locator elements 50 contact the proximal end 62 of the guide catheter 60, whereupon the locator elements 50 may be deflected proximally to the contracted condition as the locator elements 50 enter the lumen 66. Thus, as the delivery catheter 12 is advanced through the guide catheter 60, the wall of the lumen 66 may constrain or maintain the locator elements 50 in the contracted condition.

Alternatively, the locator elements 50 may be constrained in the contracted condition before being advanced into the guide catheter 60. For example, before introducing the delivery catheter 12 into the patient's body, an overlying sheath (not shown) may be provided on the distal end 16 of the delivery catheter 12, e.g., that covers the locator elements 50, stent 40, and/or balloon 22. The sheath may be provided as part of the apparatus 10 before use by the user or may be advanced over the locator elements 50, stent 40, and/or balloon 22 by the user shortly before the procedure.

In another alternative, a relatively short sleeve may be provided on the distal end 16 that constrains the locator elements 50 in the contracted condition. Along with the distal end 16 of the delivery catheter 12, the sleeve may be partially advanced into the proximal end 62 of the guide catheter 60, e.g., sufficient distance to ensure that the locator elements 50 are disposed within the guide catheter 60. The sleeve may then be withdrawn and removed from around the delivery catheter 12, leaving the locator elements 50 constrained within the guide catheter 60. For example, the sleeve may be withdrawn towards the proximal end 14 (not shown, see FIGS. 1 and 2) of the delivery catheter 12, where the sleeve may remain during the procedure. Alternatively, the sheath may include one or more weakened regions (not shown), allowing the sheath to be peeled or otherwise separated and removed entirely from around the delivery catheter 12.

Although the locator elements 50 may be biased to extend outwardly against the wall of the lumen 66, the guide catheter 60 may allow the locator elements 50 to slide freely within the lumen 66 while remaining in the contracted condition. Optionally, the locator elements 50 and/or the guide catheter 60 may include a lubricious coating to reduce friction and/or otherwise facilitate advancement through the guide catheter 60.

Returning to FIG. 4, with the distal end 64 of the guide catheter 60 against or adjacent the ostium 90, the distal end 16 of the delivery catheter 12 may be advanced from the guide catheter 60, through the ostium 90, and into the branch 94. For example, the delivery catheter 12 may be advanced until the stent 40 extends into and through the lesion 96.

Turning to FIG. 5, in one embodiment, the delivery catheter 12 may be advanced until the stent 40 is disposed distally beyond the lesion 96 within the branch 94. This step may provide the user with confidence that the stent 40 is able to be positioned within the lesion 96 before the stent 40 is expanded.

As best seen in FIG. 5A, the locator elements 50 may be at least partially exposed within the ostium 90 and/or branch 94. In addition or alternatively, the guide catheter 60 may be at least partially withdrawn, e.g., such that the distal end 64 is moved away from the ostium 90, as shown in FIG. 6, to further deploy the locator elements 50.

With continued reference to FIG. 6, as the second ends 54 of the locator elements 50 are exposed, the locator elements 50 may be biased to expand towards the enlarged condition. However, because the locator elements 50 are within the branch 94 and/or lesion 96, the locator elements 50 may remain substantially in the contracted condition, i.e., at least partially constrained by the wall of the branch 94 and/or the lesion 96 itself. In one embodiment, the locator elements 50 may be at least partially radiopaque, as described above, such that the locator elements 50 may be monitored under fluoroscopy. Thus, using fluoroscopy, the user may observe the locator elements 50 in the contracted condition shown in FIG. 6, thereby confirming the position of the distal end 16 of the delivery catheter 12, and consequently, the location of the stent 40, relative to the lesion 96.

Turning to FIG. 7, while continuing to monitor the locator elements 50, the delivery catheter 12 may be partially withdrawn, e.g., until the locator elements 50 begin to emerge from the ostium 90 into the trunk 92. As shown, the locator elements 50 may resiliently expand within the trunk 92, thereby allowing the user to monitor the position of the locator elements 50 and stent 40.

As shown in FIG. 8, when the locator elements 50 are released or exposed within the trunk 92, the locator elements 50 may fully expand, e.g., until they are substantially perpendicular to the branch 94 and/or parallel to the trunk 92. Using fluoroscopy, the user may monitor this configuration, which confirms that the stent 40 is disposed within the lesion 96 immediately adjacent the ostium 90. Alternatively, once the locator elements 50 are partially exposed within the trunk 92 and begin to expand, as shown in FIG. 7, the locator elements 50 may have sufficient spring force to direct the delivery catheter 12 proximally until the locator elements 50 are fully expanded, as shown in FIG. 8. Thus, the bias of the locator elements 50 may automatically position the delivery catheter 12 in the optimal position for deployment of the stent 40.

In addition, the full expansion of the locator elements 50 may provide tactile feedback to the user. For example, as the delivery catheter 12 is withdrawn, the user may feel the change in the spring force of the locator elements 50 as they assume the fully expanded enlarged condition within the trunk 92. In addition, if the user attempts to advance the delivery catheter 12 again, the locator elements 50 may contact the wall of the trunk 92 adjacent the ostium 90, thereby resisting further advancement and providing additional tactile feedback to the user. Optionally, the user may monitor the position of the locator elements 50 using fluoroscopy or other external imaging in addition to the tactile feedback provided by the locator elements 50, or the tactile feedback may be sufficient that external imaging is unnecessary.

Optionally, as shown in FIG. 10 and described above, in addition or alternatively to these methods, a balloon 58′ or other expandable member may be provided adjacent the locator elements 50.′ The balloon 58′ may be expanded after the locator elements 50′ are fully expanded, e.g., are directed or move out of the ostium 90 into the trunk 92. This may enhance the tactile feedback to the user if the user attempts to advance the delivery catheter 12′ distally, i.e., by increasing the resistance of the locator elements 50′ to deflecting against the ostium 90 or otherwise prolapsing.

With continued reference to FIG. 8, in yet another option, the user may rotate the delivery catheter 12 about its longitudinal axis 20 (shown in FIG. 1), if desired to orient the locator elements 50. For example, if the delivery catheter 12 includes an opposing pair of locator elements 50, the delivery catheter 12 may be rotated until the locator elements 50 reach a lowest energy state, i.e., corresponding closest to the enlarged condition. This may occur with the locator elements 50 aligned along a wall of the trunk 92, as shown in FIG. 8. In addition, if desired, the delivery catheter 12 may be directed distally and/or proximally one or more times to tactilely determine the desired location of the stent 40, e.g., corresponding to the lowest energy state of the locator elements 50. Any of these manipulations may also ensure that the stent 40 is properly positioned within the ostium 90.

Turning to FIG. 9, once the distal end 16 of the delivery catheter 12 is positioned properly within the ostium 90, the stent 40 may be expanded, e.g., by inflating the balloon(s) 22, to dilate or otherwise treat the lesion 96. Deployment of the stent 40 may be accomplished in a single step or multiple steps depending upon the configuration of the stent 40 and/or balloon(s) 22, as described in the applications incorporated by reference elsewhere herein.

For example, a single balloon 22 may be inflated to expand the stent 40 to the expanded and flared condition shown in FIG. 9. Alternatively, a proximal balloon (not shown) may be expanded to cause a proximal portion of the stent 40 to flare to engage the ostium. A distal balloon (also not shown) may then be expanded to expand a distal portion of the stent 40 and, optionally, to further expand the proximal portion of the stent 40 to dilate the lesion 96 and/or otherwise secure the stent 40 within the branch 94. In another alternative, a distal balloon may be expanded first to expand the stent 40 and dilate the lesion 96, whereupon a proximal balloon may be expanded to flare the stent 40 to its fully expanded condition, as shown in FIG. 9.

With the stent 40 fully deployed, the balloon 22 may be deflated or otherwise collapsed, and the delivery catheter 12 may be withdrawn into the guide catheter 60. If the delivery catheter 12′ of FIG. 10 is provided, the balloon 58′ may be deflected before withdrawing the delivery catheter 12.′

With continued reference to FIG. 9, optionally, the guide catheter 60 may be advanced towards or against the ostium 90 and/or against a proximal end of the stent 40 before the delivery catheter 12 is removed. This action may facilitate withdrawing the distal end 16 (e.g., the balloon(s) 22) back through the stent 40, e.g., without substantial risk of dislodging the stent 40 from the ostium 90 and/or branch 94.

As the distal end 16 of the delivery catheter 12 is withdrawn into the guide catheter 60, the locator elements 50 may contact the distal end 64 of the guide catheter 60 and be resiliently compressed as they are pulled into the lumen 66. Alternatively, a sheath may be advanced back over the distal end 16 to compress the locator elements 50 towards the contracted condition. As the locator elements 50 are compressed, the second ends 54 may be directed distally such that the locator elements 50 may extend distally, rather than proximally as during original advancement. The delivery catheter 12, guide catheter 60, and guidewire 98 may then be removed from the trunk 92 and the patient's body, leaving the stent 40 within the lesion 96. Optionally, the guidewire 98 and/or guide catheter 60 may be used to deliver one or more additional stents (not shown), if desired.

Turning to FIGS. 13 and 14, similar methods may be used to deliver a stent 40 using the delivery catheters 212, 212.′ Unlike the previous embodiments, the distal end 216, 216′ of the delivery catheter 212, 212′ may be positioned within the branch 94, e.g., through the lesion 96 with the locator elements 250, 250′ disposed adjacent the ostium 90. As the locator elements 250, 250′ are actuated to direct them from the contracted to the enlarged condition, the locator elements 250, 250′ may contact the ostium 90, thereby directing the delivery catheter 212, 212′ proximally until the locator elements 250, 250′ are positioned within or adjacent the trunk 92 and/or ostium 90. The stent 40 may be deployed as described above, whereupon the locator elements 250, 250′ may be actuated to return them to the contracted condition before removing the delivery catheter 212, 212′ from the patient's body.

Turning to FIG. 15, another embodiment of a delivery catheter 312 is shown that includes a single locator element 350. The locator element 350 includes one or more spokes or supports 351 that extend from a distal end 316 of the delivery catheter 312 to a ring or loop 355. The spoke(s) 351a on an inside bend of the delivery catheter 312 may be shorter than the spoke(s) 351b on an outside bend of the delivery catheter 312, as shown. Thus, the loop 355 may be supported eccentrically around the delivery catheter 312 in the enlarged condition.

In addition, the stiffness of the inside spoke(s) 351a may be less then the outside spoke(s) 351b. Thus, the portion of the loop 355 furthest from the delivery catheter 312 may have greater support than the portion closest to the delivery catheter 312, which may reduce the risk of prolapse of the loop 355 during use. In addition, the greater flexibility of the shorter spoke(s) 351a may provide more compliant support on an inside bend of the delivery catheter 312, which may reduce the risk of shorter spoke(s) 351a acting as a fulcrum and/or causing the locator element 350 to pull the stent 40 proximally too far relative to the branch 94 and/or lesion 96.

The delivery catheter 312 may be used to deliver a stent (not shown) carried over one or more balloons, with one balloon 322 shown for simplicity in FIG. 15. Similar to previous methods, the delivery catheter 312 may be advanced through a guide catheter 60 with the locator element 350 constrained in a contracted condition. The locator element 350 may be exposed at least partially within the ostium 90, whereupon the locator element 350 may begin to expand towards the enlarged condition. The delivery catheter 312 may be withdrawn proximally and/or advanced distally to allow the locator element 350 to fully expand and/or contact the wall of the trunk 92 surrounding the ostium 90. Once the delivery catheter 312 is properly positioned (using the locator element 350 to provide tactile feedback alone or with external imaging), a stent (not shown) may then be deployed to dilate or otherwise treat the lesion 96, similar to the previous embodiments.

Turning to FIGS. 16-16B, still another embodiment is shown of a delivery catheter 312′ that includes one or more balloons 322′ on a distal end 316′ thereof, e.g., for delivering a stent (not shown), generally similar to other embodiments described elsewhere herein. In addition, the delivery catheter 312′ includes a locator element 350′ on the distal end 316′ for locating an ostium (not shown). In this embodiment, the locator element 350′ includes a pair of struts or supports 351′ supporting a locator loop 355.′ Optionally, only a single support or more than two supports (not shown) may be provided.

In the relaxed, enlarged condition, as shown, the locator loop 355′ extends laterally relative to the delivery catheter 312′ away from a longitudinal axis 320′ on a first side of the delivery catheter 312.′ In addition, the locator element 350′ includes a constraining loop 356′ that extends laterally from the locator loop 355,′ e.g., around the distal end 316′ of the delivery catheter 312′ on a second side opposite the first side. As shown, the locator loop 355′ and the constraining loop 356′ both extend from free ends of the supports 351.′ Alternatively, the constraining loop 356′ may extend from the locator loop 355,′ e.g., adjacent the free ends of the supports 351.′

The locator loop 355′ may be substantially longer and/or extend radially and/or axially further than the constraining loop 356.′ Thus, the locator loop 355′ may be provided for contacting a vessel wall adjacent an ostium, e.g., a wall of a main vessel from which the ostium extends (not shown). For example, similar to the methods described elsewhere herein, the delivery catheter 312′ may be advanced into an ostium, and the locator element 350′ deployed within the ostium. The delivery catheter 312′ may then be withdrawn partially into the main vessel to allow the locator element 350′ to expand fully. Alternatively, the locator element 350′ may be deployed within the main vessel before advancement into the ostium.

The delivery catheter 312′ may then be advanced into the ostium until the locator loop 355′ contacts the vessel wall adjacent the ostium, thereby providing tactile feedback to the user of the location of the distal end 316′ of the delivery catheter 312.′ If the delivery catheter 312′ is advanced distally further, the locator element 350′ may bend, e.g., at the struts 351,′ directing the locator loop 355′ more transversely until the constraining loop 356′ contacts the distal end 316′ of the delivery catheter 312.′ With the constraining loop 356′ contacting the distal end 316,′ the locator loop 355′ may be restrained from bending further, thereby preventing further advancement of the delivery catheter 312′ and providing increased tactile feedback to the user.

Based upon this feedback, the user may be able to identify the relative location of the balloon(s) 322′ and any device, e.g., a stent carried thereon (not shown), relative to the ostium. The device may then be deployed within the ostium, similar to the previous embodiments. Once the device is delivered, the delivery catheter 312′ may be retracted and removed, also similar to the previous embodiments.

In other alternatives, any of the locator elements described herein may be provided on a separate device, e.g., a sheath (not shown) surrounding and/or movable relative to the distal end of any of the delivery catheters described herein. For example, the sheath may be movable axially relative to the delivery catheter to allow the locator elements to be moved relative to the stent and stent balloon(s). Thus, the sheath and/or delivery catheter may be manipulated independently relative to an ostium, e.g., while being monitored using fluoroscopy and the like, to properly position a stent relative to the ostium and/or lesion before deployment.

It will be appreciated that elements or components shown with any embodiment herein are exemplary for the specific embodiment and may be used on or in combination with other embodiments disclosed herein. In addition, although the embodiments described herein relate to delivering a stent, it will be appreciated that the locator elements and/or delivery catheters described herein may be used to complete other diagnostic and/or therapeutic procedures at or within an ostium. For example, the delivery catheter 12 may include one or more electrodes or other elements (not shown), rather than the stent 40. The locator elements 50 may facilitate positioning the electrode(s), which may be used to ablate or otherwise treat tissue at or within the ostium.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. An apparatus for locating an ostium of a body lumen, comprising:
a tubular member comprising a proximal end, a distal end sized for introduction into a body lumen, and a lumen extending between the proximal and distal ends;
an elongate member comprising a distal portion disposed within the lumen such that the distal portion may be advanced beyond the tubular member distal end; and
one or more locator elements on the distal portion, each locator element comprising a first end fixed to the distal portion and a second end free from the distal portion, each locator element being resiliently compressible to a contracted condition when the distal portion is disposed within the lumen such that the second end is disposed proximal to the first end, each locator element being resiliently expandable to an enlarged condition when fully deployed.

2. The apparatus of claim 1, wherein each locator element comprises a wire including at least one end fixed to the distal portion.

3. The apparatus of claim 2, further comprising an expandable support adjacent the at least one end for supporting the locator element in the enlarged condition.

4. The apparatus of claim 3, wherein the expandable support comprises a balloon disposed immediately proximal to the at least one end.

5. The apparatus of claim 1, wherein each locator element comprises a loop including first and second struts connecting by an intermediate portion, one end of the first and second struts being fixed to the distal portion, the intermediate portion defining the second end of the locator element.

6. The apparatus of claim 5, wherein the first and second struts extend transversely from the distal portion when the locator element expands to the enlarged condition.

7. The apparatus of claim 6, wherein the intermediate portion is disposed proximally from the first and second struts when the locator element is constrained in the contracted condition.

8. The apparatus of claim 1, further comprising a tubular prosthesis on the distal portion adjacent the one or more locator elements.

9. The apparatus of claim 1, wherein the tubular member comprises a guide catheter.

10. The apparatus of claim 1, wherein the one or more locator elements are biased to a substantially straight shape in the enlarged condition.

11. The apparatus of claim 1, wherein the one or more locator elements are biased to a curved shape in the enlarged condition.

12. The apparatus of claim 1, wherein the first end of the locator element comprises one or more supports, and wherein the second free end of the locator element comprises a loop supported by the one or more supports.

13. The apparatus of claim 12, wherein the loop is supported eccentrically around the distal portion in the enlarged condition.

14. The apparatus of claim 12, wherein the loop is oriented laterally away from the distal portion in the enlarged condition.

15. The apparatus of claim 14, wherein a constraint extends from the loop to a location opposite the distal portion to limit deflection of the loop.

16. A method for locating an ostium communicating from a main body lumen to a branch body lumen, comprising:
advancing a distal end of a delivery catheter into the main body lumen, the distal end comprising one or more locator elements constrained in a contracted condition;
advancing the distal end into the ostium until the one or more locator elements are disposed within the branch body lumen;
releasing the one or more locator elements within the branch body lumen;
partially withdrawing the distal end until the one or more locator elements at least partially emerge from the ostium and expand towards an enlarged condition; and
performing a procedure at or within the ostium based upon the position of the one or more locator elements in the enlarged condition.

17. The method of claim 16, wherein the one or more locator elements are monitored using fluoroscopy to identify when the one or more locator elements emerge from the ostium and expand towards the enlarged condition.

18. The method of claim 16, further comprising advancing the distal end after the one or more locator elements expand towards the enlarged condition, the one or more locator elements providing tactile feedback resisting further advancement when the one or more locator elements contact the main body lumen wall adjacent the ostium.

19. The method of claim 18, further comprising expanding an expandable support adjacent the one or more locator elements before advancing the distal end, the expandable support enhancing the tactile feedback provided by the one or more locator elements.

20. The method of claim 19, wherein the expandable support comprises a balloon disposed immediately proximal the one or more locator elements in the enlarged condition.

21. The method of claim 16,wherein the procedure comprises delivering a stent within at least one of the ostium and the branch.

22. The method of claim 21, wherein the ostium comprises a lesion, and wherein the one or more locator elements are released within the lesion, the lesion preventing the one or more locator elements from expanding fully towards the enlarged condition.

23. A method for locating an ostium communicating from a main body lumen to a branch body lumen, comprising:
advancing a distal end of a delivery catheter into the main body lumen, the distal end comprising one or more locator elements constrained in a contracted condition;
advancing the distal end into the ostium until the one or more locator elements are disposed within the branch body lumen;
releasing the one or more locator elements within the branch body lumen;
partially withdrawing the distal end until the one or more locator elements at least partially emerge from the ostium and expand towards an enlarged condition, whereupon the one or more locator elements automatically withdraw the distal end to accommodate expansion of the one or more locator elements towards the enlarged condition; and
performing a procedure at or within the ostium based upon the position of the one or more locator elements in the enlarged condition.

24. The method of claim 23, wherein the procedure comprises delivering a stent within at least one of the ostium and the branch.
